(19) United States
(12) Plant Patent Application Publication
(10) Pub. No.: US 2003/0221232 P1
(43) Pub. Date: Nov. 27, 2003

(54) Melastoma plant named 'Gifu Variegated'

(76) Inventor: Seiju Yamaguchi, Mizunami-shi (JP)

Correspondence Address: CHRISTIE, PARKER & HALE, LLP, 350 WEST COLORADO BOULEVARD, SUITE 500, PASADENA, CA, 91105, US

(21) Appl. No.: 10/153,089

(22) Filed: May 22, 2002

(51) Int. Cl. A01H 5/00

(52) U.S. Cl. PLT/263

(57) ABSTRACT

A new plant variety of Melastoma characterized by its Pink and White variegated new foliage, and White variegated mature foliage.

### BACKGROUND OF THE INVENTION

The present invention relates to a new and distinct variety of Melastoma sp. L. and was discovered as a branch sport of an unnamed variety of Melastoma sp. (unpatented) in Mizunami-shi, Gifu Prefecture, Japan by the originator, Mr. Seiju Yamaguchi. The varietal denomination of the new variety is ‘GIFU VARIEGATED’.

### SUMMARY OF THE INVENTION

The new variety was discovered as a branch sport, and differs from other cultivars known to the inventor by its Pink and White variegated new foliage, and White variegated mature foliage. Asexual reproduction of the new variety by stem cuttings in Gifu Prefecture, Japan; Lewisberry, Pa.; and Vacaville, Calif.; have confirmed that the distinctive characteristics of the new variety are stable and transmitted to succeeding generations, and the new variety reproduces true to type.

### COMPARISON WITH PARENT

‘GIFU VARIEGATED’ is distinguished from its parent and all other varieties of Melastoma of which I am aware by its Pink and White variegated new foliage, and White variegated mature foliage.

### BRIEF DESCRIPTION OF ILLUSTRATION

The accompanying photographic illustration shows a plant of the new cultivar as true to color as is reasonably possible to make in an illustration of this character.

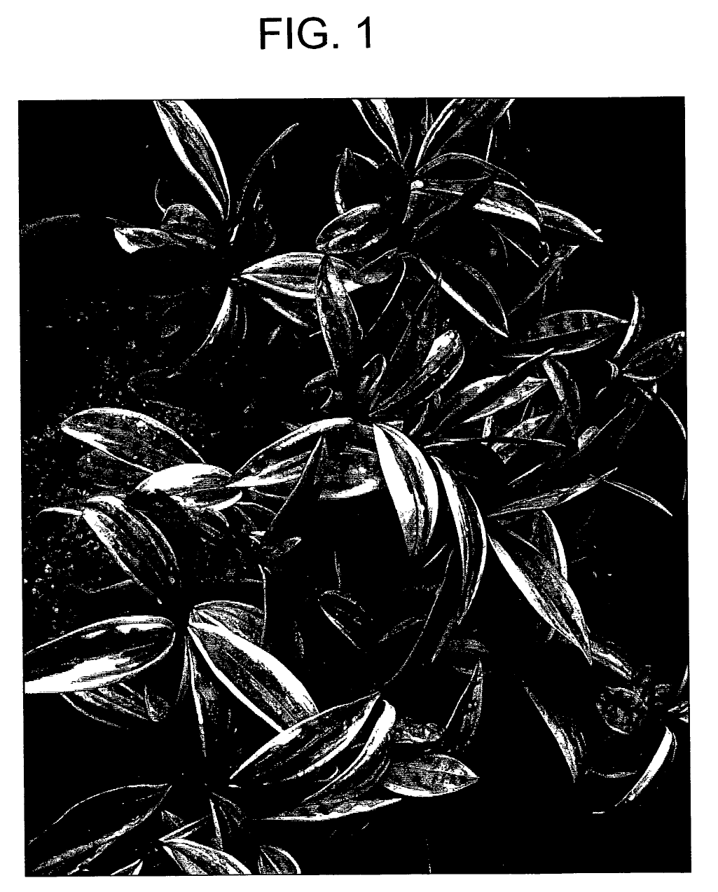

FIG. 1 illustrates the distinctive foliage variegation of the new variety.

### DESCRIPTION OF THE NEW VARIETY

Melastoma ‘GIFU VARIEGATED’ has not been observed under all possible environmental, cultural and light conditions. The following observations and descriptions are of plants grown in Vacaville, Calif. and Lewisberry, Pa. Phenotypic expression may vary with light intensity, cultural, and environmental conditions. In this description, color references are to The Royal Horticultural Society Colour Chart (2001) and terminology used in the color descriptions herein refers to plate numbers in this color chart. As with many plants in the Melastomataceae family phenotypic characteristics can be dramatically different in terms of growth morphology.

### CLASSIFICATION

Botanical: Melastoma L. ‘GIFU VARIEGATED’.

Parentage: Branch sport of unnamed plant of Melastoma.

Propagation: Stem cuttings.

Time to initiate roots.—About 14 days at 22 C.

Time to develop roots.—About 42 days at 22 C.

Root appearance.—Fibrous, freely branching.

### PLANT

Size: Mature plant: Compact to 60 cm tall and wide. Observed: 18 cm.

Branching:

Observed quantity.—6.

Branch size observed.—15 cm.

Branch diameter.—2 mm.

Branch texture.—Scabrous; irregular tiny scale-like hairs.

Branch coloration.—Young stems: Near Red Group 53B. Mature stems: Near Greyed-Orange Group 177A to 177B.

Habit: Upright, somewhat outwardly spreading and open habit; sub-shrub.

### LEAF

Shape: Nearly elliptic to elliptic.

Apex: Acute to acuminate.

Base: Attenuate.

Margin: Entire, revolute.

Texture: Smooth.

Substance: Firm.

Pattern of variegation: Broadly marginal with occasional irregular stripes invading the center of leaf, occasionally following the midrib.

Leaf color: Varies with intensity of sunlight and cultural conditions; the following color readings were taken on Dec. 14, 2001, in Vacaville, Calif. of a specimen grown in a 1-gallon nursery container, in an unheated greenhouse at an average temperature of 70° F.

Young leaves.—Green Sections Upper Side: Near Green Group 146B. Underside: Near Yellow-Green Group 148B. Variegated Sections Upper Side: Near Red Group 51C. Underside: Near Red Group 51C.

Mature foliage.—Green Sections Upper Side: Plant displays 2 segments of variegation; near Yellow-Green Group 148C and near Green Group 138B. Underside: Near Yellow-Green Group 148D; bottom of leaf margin on mature foliage exhibits coloration near Red Group 51C.

Variegated sections.—Upper Side: Near White Group N155B. Underside: Near White Group N155D.

Lateral branches:

Quantity.—Freely branching; pinching enhances lateral branch development.

Diameter.—About 5.5 mm.

Texture.—Scabrous; irregular tiny scale-like hairs.

Venation pattern.—Parallel; 3 veins; impressed on upper side, raised on underside.

Petioles:

Size.—1 to 2 cm long.

Coloration.—Upper Surface: Near Red-Purple Group 61B. Lower Surface: Near Red Group 53B.

Internodes.—2.5 cm.

Leaf size.—7.5 to 9.5 cm long; 2 to 3 cm wide.

Hardiness: USDA Zone 10 (30° to 40° F.)

Vigor: Moderate.

Pests/Diseases: None noted.

### INFLORESENCE

Bloom period: May through September.

Flower arrangement: Terminal cymes.

Flower form: Radial, 4-petaled.

Flower size: 3.5 to 4 cm in diameter.

Lastingness of individual blooms: 5 to 7 days.

Fragrance: None noted.

Petals:

Coloration.—Upper Surface: Near Purple-Violet Group N82B.

Length/Width.—1.75 to 2 cm long; 1 to 1.5 cm wide.

Buds:

Coloration.—Near Purple-Violet Group N82D.

I claim:

1. A new plant variety of Melastoma of the variety substantially as shown and described.
